(19) United States
(12) Patent Application Publication
(10) Pub. No.: US 2007/0281960 A1
(43) Pub. Date: Dec. 6, 2007

(54) Anti-Histaminic Composition

(76) Inventors: Amar Lulla, Maharashtra (IN); Geena Malhotra, Maharashtra (IN); Sankarnarayanan Anand, Tamil Nadu (IN)

Correspondence Address: CONLEY ROSE, P.C., 5601 GRANITE PARKWAY, SUITE 750, PLANO, TX, 75024, US

(21) Appl. No.: 11/572,175

(22) PCT Filed: Jul. 18, 2005
(86) PCT No.: PCT/GB05/02828

§ 371 Date: Jul. 2, 2007

(30) Foreign Application Priority Data
Jul. 16, 2004 (IN) 765/MUM/2004

(51) Int. Cl. A61K 31/4545; A61P 37/08

(52) U.S. Cl. 514/290

(57) ABSTRACT

A stable pharmaceutical composition comprises desloratadine or a pharmaceutically acceptable salt, solvate, derivative, polymorph, hydrate or enantiomer thereof, and a carrier comprising at least one polyol. Preferred compositions comprise, in addition to desloratadine, from 50 to 80% polyol, from 5 to 15% disintegrating agent, and from 0.01 to 0.5% antioxidant and/or a chelating agent, all by weight of the composition. The polyol may be used to stabilise a pharmaceutical composition comprising desloratadine. A process for making a stable pharmaceutical composition comprising desloratadine and one or more polyols, optionally together with other pharmaceutically acceptable excipients comprises blending the ingredients and formulating them so as to form said composition.

The present invention relates to a pharmaceutical composition comprising desloratadine or a pharmaceutically acceptable salt, solvate, derivative, polymorph, hydrate or enantiomer thereof, to a process for preparing such a composition, and to therapeutic uses thereof.

Desloratadine is DCL or Descarbonylethoxyloratadine or 8-chloro-6,11-dihydro-11-(4-piperidylidene)-5-H-benzo [5,6] cyclohepta [1,2-b] pyridine and possesses antihistaminic properties with substantially no sedative properties.

Desloratadine is an active metabolite of loratadine. It is an oral, long-acting antihistamine that is similar chemically to loratadine. It is 50-fold more potent in-vitro and 10-fold more potent in-vivo than loratadine. It is used to treat the symptoms caused by histamine. Histamine is a chemical that is responsible for many of the signs and symptoms of allergic reactions, for example, swelling of the lining of the nose, sneezing, and itchy eyes. Histamine is released from histamine-storing cells (mast cells) and then attaches to other cells that have receptors for histamine. The attachment of the histamine to the receptors causes the cell to be “activated”, releasing other chemicals, which produce the effects that we associate with allergy.

Desloratadine is a long-acting tricylic histamine agonist with selective H₁-receptor histamine antagonist activity. It blocks the H₁ receptor for histamine and thus prevents activation of H₁ receptor-containing cells by histamine. Desloratadine inhibits histamine release from human mast cells in vitro. Desloratadine does not readily enter the brain from the blood and, therefore, causes less drowsiness (sedation). It is a member of a small family of non-sedating antihistamines, which includes loratadine, cetrizine, and azelastine. Use of Desloratadine to relieve hay fever and allergy symptoms, including sneezing; runny nose; and red, itchy, tearing eyes, hives has been reported in the literature.

Desloratadine, a non-sedating antihistamine is disclosed in U.S. Pat. No. 4,659,716. This US patent also discloses methods of making loratadine, pharmaceutical compositions and methods of using the compositions to treat allergic reactions in mammals.

U.S. Pat. No 5,595,997 discloses that desloratadine, a metabolic derivative of loratadine, is used for the treatment of allergic rhinitis, and other disorders, and also avoids the concomitant liability of adverse side-effects associated with other non-sedating antihistamines.

WO0310143 describes pharmaceutical compositions comprising an antihistamine with or without a leukotriene inhibitor for intra nasal delivery to the nasal mucosa.

Methods for treating urinary incontinence comprising administering a therapeutically effective amount of desloratadine, or a pharmaceutically acceptable salt thereof is disclosed in U.S. Pat. No. 5,939,426.

U.S. Pat. No 6,100,274 discloses a pharmaceutical composition containing desloratadine (DCL), and a DCL-protective amount of a pharmaceutically acceptable basic salt such as calcium dibasic phosphate, plus an amount of at least one disintegrant, preferably two disintegrants such as microcrystalline cellulose and starch sufficient to provide dissolution of at least about 80% by weight of the pharmaceutical composition in about 45 minutes and suitable for oral administration to treat allergic reactions in mammals such as man are disclosed. The use of the basic salt is disclosed as being essential to the stability of the composition. The compositions are said to be substantially free of discoloration and to contain less than 1% by weight of N-Formyl descarbonylethoxyloratadine after storage at 25° C./60% RH for at least 24 months. By contrast, formulations without a basic salt and containing excipients such as mannitol, lactose, and magnesium stearate were all found to be unstable.

Desloratadine is known to be a sensitive molecule that is found to get discoloured and decompose due to harsh temperature and humidity conditions. As taught in U.S. Pat. No. 6,100,274, it is also found to discolor and decompose with incompatible excipients. The discoloration and decomposition at elevated temperature and humidity is accelerated when combined with incompatible (or) highly reactive excipients.

We have recognised that there remains a need for formulations of desloratadine which do not discolor and which are substantially free of desloratadine (DCL) decomposition products, even under accelerated stability conditions.

An object of the present invention is to provide a formulation of desloratadine which does not discolour and is substantially free from decomposition products of DCL, especially N-formyl desloratadine, and is stable even at accelerated conditions. The wording “substantially free from decomposition products” as used herein denotes minimal impurities, in particular N-formyl desloratadine.

We have now found that, surprisingly, desloratadine can be satisfactorily stabilised without, in particular, the need to resort to the use of basic salts.

Accordingly, in a first aspect, the present invention provides a stable pharmaceutical composition comprising desloratadine or a pharmaceutically acceptable salt, solvate, derivative, polymorph, hydrate or enantiomer thereof, and a carrier comprising at least one polyol.

In another aspect, the present invention provides a pharmaceutical composition comprising desloratadine or a pharmaceutically acceptable salt, solvate, derivative, polymorph, hydrate or enantiomer thereof, and a carrier comprising at least one polyol, excluding a tablet comprising (mg/tablet) desloratadine (2.5), microcrystalline cellulose (10), mannitol (71.5), pregelinized starch (15) and magnesium stearate (1).

The invention also provides a pharmaceutical composition comprising desloratadine or a pharmaceutically acceptable salt, solvate, derivative, polymorph, hydrate or enantiomer thereof, and a carrier comprising at least one polyol, wherein the composition does not contain one or more of the following ingredients: sugar, such as lactose; stearic acid and derivatives thereof, including sodium stearyl fumerate, magnesium stearate, calcium stearate; aspartame; zinc ascorbate; ascorbyl palmitate.

In another aspect, there is provided a pharmaceutical composition consisting essentially of desloratadine or a pharmaceutically acceptable salt, solvate, derivative, polymorph, hydrate or enantiomer thereof and one or more polyols. Preferably, the composition further consists of one or more of a disintegrating agent, an antioxidant, a chelating agent, and a lubricant.

The compositions of the present invention may optionally be coated. Any suitable coating may be used and coating may, in some instances, improve stability. Film coatings are preferred, but other suitable coatings include wax and sugar coatings (excluding lactose) may be used.

In another aspect, the invention provides the present compositions for use as medicaments, in particular for treating conditions responsive to administration of an antihistamine.

According to the present invention there is also provided a stable pharmaceutical composition comprising desloratadine or a pharmaceutically acceptable salt, solvate, derivative, hydrate, polymorph or enantiomer thereof and a stabilizing amount of one or more polyols with one or more pharmaceutically acceptable excipients such that the total impurities amount to less than 1.5% by weight of the active. Suitably, this means when the composition is stored at 25° C. and about 60% relative humidity for a period of at least 24 months. The impurities consist essentially of degradation/decomposition products of the active material.

Desloratadine is a highly reactive compound which when exposed to high temperature and humid storage condition degrades and changes to pink colour. Therefore, extreme caution needs to be taken when formulating desloratadine with various excipients. We have found that desloratadine is incompatible with common excipients such as lactose and magnesium stearate, microcrystalline stearate and degrades and changes to a pink colour. This decomposition is further accelerated under high temperature and humid conditions.

According to U.S. Pat. No 6,100,274, the main degradant of desloratadine is N-formyl descarbonylethoxyloratadine or N-formyl desloratadine. The formation of the degradation product increases at higher temperature and humidity conditions.

The term “desloratadine” is used herein in a broad sense to include not only desloratadine per se, but also pharmaceutically acceptable salts, solvates, derivatives, polymorphs, hydrates or enantiomers thereof. Polymorphic Forms I and II are preferred forms. These can be used alone or mixtures of Form I and Form II may be used.

By “stable” composition we mean compositions that show compliance to physicochemical parameters, for example which do not show any significant discolouration and which are substantially free of desloratadine (DCL) decomposition products and suitably, the content of drug does not fall below 95% by weight of the active, and dissolution is not less than 80% by weight of the composition during the specified shelf life. Preferably, this applies even after prolonged (eg 12 or 24 months) storage at ambient conditions. Suitably, compositions of the invention contain total decomposition products (including N-formyl desloratadine) less than 1.5% by weight of the active. Preferably, the amount of N-formyl impurity is less than 0.75%, preferably less than 0.6%, by weight of the active after storage at 40° C. and 75% relative humidity for at least 1 month, preferably 4 months, more preferably for at least 6 months. Preferably, compositions of the invention contain N-formyl desloratadine less than 0.75%, ideally less than 0.6%, preferably less than 0.5%, by weight of the active after storage at 25° C. and 60% relative humidity. Preferably, the period of storage is for at least 12 months, more preferably at least 18 months, and most preferably at least 24 months, at 25° C. and 60% relative humidity. We have found that it is possible to provide such stable compositions by using a polyol in combination with the active, without the need to use additional stabilising means such as basic salts.

Accordingly, the invention also provides the use of a polyol to stabilise a pharmaceutical composition comprising desloratadine.

The carrier for the active in the compositions of the invention may comprise one or more polyols alone, but will preferably also include other excipients provided that these do not adversely affect the stability of the active. Compounds which show an acidic pH in water are preferably excluded.

Surprisingly, polyols or sugar alcohols like mannitol, sorbitol, maltitol, xylitol, erythritol and the like (which are used normally as fillers), when used as carriers the degradation and discoloration of the desloratadine is reduced substantially.

Polyol (also known as sugar alcohol, polyhydric alcohol) is a hydrogenated form of carbohydrate, whose carbonyl group (aldehyde or ketone), reducing sugar) has been reduced to a primary or secondary hydroxyl group. They are commonly used as artificial sweeteners. Some of the preferred polyols are mannitol, sorbitol, xylitol, maltitol, lactitiol, erythritol.

Mannitol is preferably D-mannitol. It is a hexahydric alcohol related to mannose and is isomeric with sorbitol. Mannitol occurs as a white, odourless, crystalline powder, or free flowing granules.

Sorbitol is preferably D-sorbitol. It occurs as a white or almost white, crystalline powder.

Xylitol is preferably meso-xylitol. Xylitol occurs as white crystalline powder or crystals.

Maltitol is preferably D-maltitol. It occurs as a white, crystalline powder.

Lactitol is preferably D-glucitol. Lactitol is a white crystalline powder.

Erythritol is preferably meso-Erythritol. It occurs as a white or almost white, crystalline powder or free-flowing granules.

The concentration of the said polyol added is suitably from 1-95% by weight of the composition. More preferably, 15-85% by weight of the composition is used. A particularly preferred range is from 50 to 80% by weight, as very good stabilisation is achieved within this range.

The present invention provides a final formulation that is substantially free of impurities, both initially and after prolonged storage. The present invention limits the total impurities to less than 1.5% by weight of the active.

Preferably, the compositions of the invention comprise one or more disintegrating agents. When combined with suitable disintegrants, the formulations show faster disintegration and thereby faster dissolution.

Any suitable disintegrating agent may be used, provided it does not adversely affect the stability of the active. Suitable disintegrating agents include one or more of starch, modified starch such as partially gelatinised starch, sodium starch glycolate, hydroxypropylcellulose (HPC), low substituted hydroxypropylcellulose (L-HPC) or croscarmellose sodium. A preferred disintegrant is low substituted hydroxypropylcellulose. The disintegrating agent is preferably used at in an amount of from 0.5 to 30% by weight of the composition. A range of 5 to 15% by weight is particularly suitable.

The compositions of the invention preferably also contain an antioxidant and/or a chelating agent. The presence of one or more of these compounds can serve to assist in reducing decomposition and/or discoloration of the active. An antioxidant is particularly preferred. The antioxidant and/or chelating agent may be present in an amount of from 0.01% to 1% by weight of the formulation.

A particularly preferred range is from 0.01 to 0.5% by weight of the composition.

Any suitable antioxidant may be used, but preferred antioxidants include one or more of butylated hydroxy anisole, butylated hydroxy toluene, propyl gallate, sodium metabisulfite, sodium ascorbate, and vitamin E acetate. Antioxidants at a concentration of about 0.01 to 1% w/w of a tablet more preferably 0.01 to 0.5% w/w of a tablet can, for example, be used. The most preferred antioxidant is sodium metabisulphite. The chelating agent may, for example, be disodium edetate.

A preferred composition of the invention is a stable pharmaceutical composition comprising desloratadine or a pharmaceutically acceptable salt, solvate, derivative, hydrate, polymorph or enantiomer thereof, from 50 to 80% polyol, from 5 to 15% disintegrating agent, and from 0.01 to 0.5% of an antioxidant and/or a chelating agent, all by weight of the composition. Preferably an antioxidant is used, and the active is preferably present in an amount of from 2.5 to 5 mg (for example, in a tablet, although any suitable dosage form may be used). Preferably, a lubricant is also included.

Other conventional pharmaceutical excipients may be included in the compositions of the invention as required depending upon the nature of the formulation and provided they do not adversely affect the stability of the active.

Other standard pharmaceutical excipients which may be used include one or more of but are not limited to: 1) Fillers like starch etc. 2) Binders: gelatin, pregelatinised starch, hydroxypropyl methylcellulose (HPMC), ethyl cellulose, hydroxy ethylcellulose, hydroxy propylcellulose, etc. 3) Antiadherents, lubricants and glidants like talc, hydrogenated castor oil, hydrogenated vegetable oil, siliconised talc, sucrose esters, silicone or simethicone oil, anhydrous colloidal silica etc, or mixtures of two or more thereof. The antiadherents, lubricants and glidants are preferably used in an amount of from 0.5 to 35% by weight of the composition. Pharmaceutical excipients for solutions or suspensions include sorbitol, propylene glycol, lactitiol, maltitol etc.

The finished product of the present invention can be of any suitable dosage form, for example tablets, capsules, pellets, powders, solutions or suspensions. Preferably the composition is a solid composition, especially a tablet, capsule, or pellet and the like.

In another preferred embodiment the compositions of the present invention may be coated. The coating may be a film coating, sugar coating (other than lactose) or a wax coating, for example, although other suitable coatings may be used. Film coated tablets are preferred. Preferably the film coat is lactose free. The film coating composition may, for example, be any suitable film coat and may, for example, be film coating redimix or may contain one or more conventional film forming materials like hydroxypropyl methylcellulose, ethyl cellulose, and hydroxyethylcellulose. Plasticizers, opacifiers, colouring agents, and drying agents etc may also be included. Opacifiers may, for example, be chosen from titanium dioxide, lake of various dyes and iron oxides eg iron oxide red. Colouring agents may, for example, be suitable soluble colour dyes as well as soluble lakes.

Plasticizers may also be included and suitable compounds include one or more of propylene glycol, polyethylene glycol, triethyl citrate, dibutyl sebacate, triacetin, and isopropyl myristate. Suitable vehicles may include for example one or more of water, hydroalcoholic or non-aqueous solvents.

The wax coating may include for example bees wax, gum acacia, carnuba wax, branded grades like opagloss or mixtures thereof using suitable vehicles, for example, isopropyl alcohol, methylene chloride, chloroform or mixtures thereof

one embodiment the formulation of the present invention may be in the form of a tablet. Tablet formulations of the present invention comprising polyol as an excipient along with other conventional excipients were exposed in a petri dish at 40° C./75% RH for one month. Even after one month, the tablets were found to be free of discolouration and remained white in colour. Upon analysis, the formulations did not show any further rise in degradation level. Stability data for these is given in Table 1.

Tabletting may be done using conventional methods. We prefer to use direct compression. Dry granulation by compaction or slugging may be employed. Non-aqueous granulation with cellulose polymer, such as ethylcellulose, low-substituted hydroxypropylcellulose or hydroxypropylmethyl cellulose as binder, using isopropyl alcohol and methylene dichloride as solvents may also be used. Disodium EDTA may be included.

The compositions of the invention may be coated or uncoated, for example coated or uncoated tablets. For coated compositions, an antioxidant may be included although can be left out if desired.

The invention therefore provides a stable coated pharmaceutical composition comprising desloratadine and one or more polyols. Preferably, the composition is a coated tablet. An antioxidant may optionally be present. The coating is suitably film coated and preferably lactose free. It may be a sugar coating (other than lactose) eg. sugar (pharma grade) or a wax coating, for example, although other suitable coatings may be used.

The invention also provides a stable uncoated pharmaceutical composition comprising desloratadine, one or more polyols and one or more antioxidants.

In another aspect, the invention provides a process for making a stable pharmaceutical tablet composition comprising desloratadine and one or more polyols, optionally together with other pharmaceutically acceptable excipients, which process comprises blending the ingredients and compressing to form a tablet.

The therapeutically effective amount of desloratadine for oral administration may vary, for example, from about 2.5 to 20 mg per day, more preferably from about 5 to 10 mg per day. The most preferred amount is 5 mg, once a day.

It will be readily apparent to one skilled in the art that varying substitutions and modifications may be made to the invention disclosed herein without departing from the scope and spirit of the invention. Thus, it should be understood that although the present invention has been specifically disclosed by preferred embodiments and optional features, modification and variation of the concepts herein disclosed may be resorted to by those skilled in the art, and that such modifications and variations are considered to be falling within the scope of the invention.

The following examples are for the purpose of illustration of the invention only and are not intended in any way to limit the scope of the invention.

### EXAMPLE 1

A tablet by direct compression with mannitol (DC grade)

| SR No | Ingredients | Mg/Tab |
|---|---|---|
| 1. | Desloratadine | 5.0 |
| 2. | Mannitol | 10.0 |
| 3. | Mannitol (DC grade) | QS to 140 |
| 4. | Low substituted Hydroxy | 10.0 |
| | propyl cellulose (L-HPC) | |
| 5. | Talc | 10.0 |
| | Total | 140.0 |

Manufacturing Procedure:

Desloratadine and mannitol were cosifted to form a drugmix. A blend of the drugmix L HPC and DC grade mannitol was made and lubricated and compressed to form tablets.

### EXAMPLE 2

A tablet by wet granulation with mannitol

| SR No | Ingredients | Mg/Tab |
|---|---|---|
| | Dry Mix | |
| 1. | Desloratadine | 5.0 |
| 2. | Mannitol | QS to 140 |
| 3. | Low substituted Hydroxy | 10.0 |
| | propyl cellulose (L-HPC) | |
| | Binder | |
| 4. | Hydryoxy propylcellulose | 3.0 |
| 5. | Distilled water | QS |
| | Lubrication | |
| 6. | Talc | 10.0 |
| | Total | 140.0 |

Manufacturing Procedure:

Desloratadine, mannitol and L HPC were cosifted and granulated with hydroxypropyl cellulose. The wet mass so obtained was dried and passed through mesh. It was further lubricated with talc and compressed to form tablets.

### EXAMPLE 3

A tablet by direct compression with mannitol (DC grade) and antioxidant

| SR No | Ingredients | Mg/Tab |
|---|---|---|
| 1. | Desloratadine | 5.0 |
| 2. | Mannitol | 10.0 |
| 3. | Sodium metabisulfite | 0.5 |
| 4. | Mannitol (DC grade) | QS to 140 |
| 5. | Low substituted Hydroxy | 10.0 |
| | propyl cellulose (L-HPC) | |
| 6. | Talc | 10.0 |
| | Total | 140.0 |

Manufacturing Procedure:

Desloratadine, sodium metabisulfite and mannitol were cosifted to form Drug mix. A blend of Drugmix, L HPC and DC grade mannitol was lubricated and compressed to form tablets.

### EXAMPLE 4

A tablet by direct compression with mannitol (DC grade) and further coating with a coat composition containing lactose.

| SR No | Ingredients | Mg/Tab |
|---|---|---|
| 1. | Desloratadine | 5.0 |
| 2. | Mannitol | 15.0 |
| 3. | Mannitol (DC grade) | QS to 140 |
| 4. | Low substituted Hydroxy | 15.0 |
| | propyl cellulose (L-HPC) | |
| 5. | Talc | 7.0 |
| 6. | Hydrogenated castor oil | 10.0 |
| | Total | 140.0 |
| | Coating composition | |
| 7. | Hydroxy propyl | 2.5 |
| | methylcellulose | |
| 8. | Propylene Glycol | 0.5 |
| 9. | Lactose monohydrate | 0.5 |
| 10. | Talc | 0.5 |
| 11. | Titanium dioxide | 0.75 |
| 12. | Iron oxide Red | 0.25 |
| | Total | 5.0 |

Manufacturing Procedure:

Desloratadine and mannitol were cosifted to form Drug mix. A blend of Drug mix, L HPC and DC grade mannitol was lubricated and compressed to form tablets. The tablets were coated with the above coating composition.

### EXAMPLE 5

A tablet by direct compression with Mannitol (DC grade) and coating with a coat composition without lactose.

| SR No | Ingredients | Mg/Tab |
|---|---|---|
| 1. | Desloratadine | 5.0 |
| 2. | Mannitol | 15.0 |
| 3. | Mannitol (DC grade) | QS to 140 |
| 4. | Low substituted Hydroxy | 15.0 |
| | propyl cellulose (L-HPC) | |
| 5. | Talc | 7.0 |
| 6. | Hydrogenated castor oil | 10 |
| | Total | 140.0 |
| | Coating composition | |
| 7. | Hydroxy propyl | 3.0 |
| | methylcellulose | |
| 8. | Propylene Glycol | 0.5 |
| 9. | Talc | 0.5 |
| 10. | Titanium dioxide | 0.75 |
| 11. | Iron oxide Red | 0.25 |
| | Total | 5.0 |

Manufacturing Procedure:

Desloratadine and mannitol were cosifted to form Drug mix. A blend of Drugmix, L HPC and DC grade mannitol was lubricated and compressed to form tablets. The tablets were coated with the above coating composition.

### EXAMPLE 6

A tablet by wet granulation using conventional excipients without using any polyol

| SR No | Ingredients | Mg/Tab |
|---|---|---|
| | Dry mix | |
| 1. | Desloratadine | 5.0 |
| 2. | Starch | 5.0 |
| 3. | Microcrystalline cellulose | QS to 140 |
| 4. | Crospovidone | 5.0 |
| | Binder | |
| 5. | Starch | 3.0 |
| 6. | Distilled water | QS |
| | Lubrication | |
| 7. | Crospovidone | 5.0 |
| 8. | Talc | 5.0 |
| | Total | 140.0 |

Manufacturing Procedure:

Desloratadine, starch, crospovidone and microcrystalline cellulose were cosifted to form a blend which was granulated with Starch paste. The wet mass obtained was dried and passed through mesh and lubricated with talc and compressed to form tablets.

The tablets obtained from Examples 1 to 6 were exposed in an open Petri dish at 25° C./60% RE and 40° C./75% RH for 1 month and checked for discoloration, content of desloratadine and the impurity levels. The results are given in Table I.

TABLE 1

| | | Initial | | | | 1 Month 25° C./60% RH | | | |
|---|---|---|---|---|---|---|---|---|---|
| | | | | Impurity (%) | | | | Impurity (%) | |
| | | | | Single | | | | Single | |
| Example | | Color/ | Assay | Max | | Color/ | Assay | Max | |
| No | Examples | Description | (%) | Unknown | Total | Description | (%) | Unknown | Total |
| 1. | DCL + mannitol | White | 99 | 0.2 | 0.363 | White | 101 | 0.18 | 0.35 |
| 2. | DCL + mannitol | White | 101 | 0.1 | 0.21 | White | 99 | 0.19 | 0.38 |
| 3. | DCL + mannitol + | White | 99 | 0.19 | 0.29 | White | 99 | 0.20 | 0.28 |
| | Antioxidant | | | | | | | | |
| 4. | DCL + mannitol and | White | 102 | 0.131 | 0.318 | White | 98 | 0.155 | 0.30 |
| | coating (with | | | | | | | | |
| | coating | | | | | | | | |
| | composition | | | | | | | | |
| | containing lactose | | | | | | | | |
| 5. | DCL + mannitol and | White | 100 | 0.12 | 0.22 | White | 101 | 0.16 | 0.29 |
| | coating (with | | | | | | | | |
| | coating | | | | | | | | |
| | composition not | | | | | | | | |
| | containing lactose) | | | | | | | | |
| 6. | DCL without any | White | 99 | 0.10 | 0.30 | Pale pink | 99 | 0.31 | 0.55 |
| | polyol | | | | | | | | |

| | 1 Month 40° C./75% RH | | | |
|---|---|---|---|---|
| | | | Impurity (%) | |
| | | | Single Max | |
| Example No | Color/Description | Assay (%) | Unknown | Total |
| 1. | Very pale | 100 | 0.16 | 0.40 |
| | pink | | | |
| 2. | Very pale | 98 | 0.20 | 0.40 |
| | pink | | | |
| 3. | White | 101 | 0.28 | 0.34 |
| 4. | White | 98 | 0.21 | 0.482 |
| 5. | White | 100 | 0.18 | 0.310 |
| 6. | Pink | 98 | 0.55 | 1.42 |

Note:
for colour and description of coated tablets the tablets exposed for 1 month were broken and observed.
1) Comparing examples, 1 and 2 with 6 it can be seen that Polyols stabilize Desloratadine formulation and substantially limit the impurity levels.
2) Comparing examples 1 and 2 with 3 it can be seen that use of antioxidants reduces discoloration of Desloratadine formulation.
3) Comparing examples 4 with 5 it can be seen that coating with lactose free composition enhances the stability and further reduces discoloration of Desloratadine formulation

### EXAMPLE 7

Capsule Form:

| SR No | Ingredients | Mg/Tab |
|---|---|---|
| 1. | Desloratadine | 5.0 |
| 2. | Mannitol | 10.0 |
| 3. | Sodium metabisulphite | 0.5 |
| 4. | Mannitol (DC grade) | QS to 140 |
| 5. | Low substituted Hydroxy | 10.0 |
| | propyl cellulose (L-HPC) | |
| 6. | Talc | 10.0 |
| | Total | 140.0 |

Desloratadine, mannitol and sodium metabisulphite were cosifted to form a drugmix. The drugmix, L-HPC and DC grade mannitol was blended and lubricated with talc and then encapsulated.

### EXAMPLE 8

Tablet by direct compression.

| Sr. No | Ingredients | Qty/Tab. (mg) |
|---|---|---|
| | Dry Mix | |
| 1. | Desloratadine (Form I) | 5.0 |
| 2. | Mannitol | 10.0 |
| 3. | Mannitol (DC grade) | 96.5 |
| 4. | Low substituted hydroxy | 15.0 |
| | propyl cellulose (L-HPC) | |
| 5. | Silica colloidal anhydrous | 1.5 |
| | Lubrication | |
| 6. | Siliconised Talc (3% w/w) | 16.0 |
| 7. | Castor Oil Hydrogenated | 12.0 |
| | (Powder grade) | |
| | Total | 156.00 |

For Film Coating:

| Sr. | Ingredients | Mg/Tab |
|---|---|---|
| 1 | Film coating Redimix | 4.0 |
| 2 | Purified water | q.s. |

Desloratadine and mannitol were cosifted to form Drug premix. The Drug premix was further blended with L-HPC, silica colloidal anhydrous and DC grade mannitol and this was further lubricated with siliconised talc and castor oil hydrogenated and compressed to form tablets. The tablets were coated with the above film coating composition.

| SR No | Ingredients | Mg/Tab |
|---|---|---|
| 1. | Desloratadine | 5.0 |
| 2. | Mannitol | 10.0 |
| 3. | Sodium metabisulphite | 0.5 |
| 4. | Sorbitol | 50 |
| 5. | Mannitol (DC grade) | QS to 140 |
| 6. | Low substituted Hydroxy | 10.0 |
| | propyl cellulose (L-HPC) | |
| 7. | Talc | 10.0 |
| | Total | 140.0 |

Tablet made by direct compression in accordance with procedure in earlier Examples.

### EXAMPLE 10

| Sr. No. | Ingredients | Qty/kg |
|---|---|---|
| 1. | Desloratadine | 5.00 |
| 2. | Sorbitol (DC grade) | q.s. |
| 3. | Sodium Metabisulphite | 0.5 |
| 4. | Low substituted hydroxy | 20.00 |
| | propyl cellulose (L-HPC) | |
| 5. | Siliconised Talc | 12.00 |
| 6. | Starch | 20.00 |
| | Total | 140.00 |

a) Sift & mix all above ingredients in suitable blender
b) Compress using suitable tooling

### EXAMPLE 11

| Sr. No. | Ingredients | Qty/kg |
|---|---|---|
| 1 | Desloratadine | 5.00 |
| 2. | Sorbitol (DC grade) | q.s. |
| 3. | Sodium Metabisulphite | 0.5 |
| 4. | Low substituted hydroxy | 20.00 |
| | propyl cellulose (L-HPC) | |
| 5 | L-HPC | 3.00 |
| 6. | Isopropyl alcohol | q.s. |
| 7. | Methylene chloride (CH₂CL₂) | q.s. |
| 8. | Disodium EDTA | 0.2 |
| 9 | Siliconised Talc | 12.00 |
| 10 | Starch | 20.00 |
| | Total | 140.00 |

Desloratadine, DC grade sorbitol were mixed to form a dry mix. A binder solution of L-HPC in isopropyl alcohol and methylene chloride were prepared. The dry mix was granulated using the binder solution. The other excipients and lubricants were added and the resulting blend was compressed using suitable tooling.

### EXAMPLE 12

Tablet by direct compression.

| Sr. No | Ingredients | Qty/Tab. (mg) |
|---|---|---|
| | Dry Mix | |
| 1. | Desloratadine (Form I) | 5.0 |
| 2. | Mannitol | 10.0 |
| 3. | Mannitol (DC grade) | 96.5 |
| 4. | Low substituted hydroxy | 15.0 |
| | propyl cellulose (LHPC) | |
| 5. | Silica colloidal anhydrous) | 1.5 |
| | Lubrication | |
| 6. | Siliconised Talc (3% w/w) | 16.0 |
| 7. | Castor Oil Hydrogenated | 12.0 |
| | (Powder grade) | |
| | Total | 156.00 |

Coating

| Sr. | | |
|---|---|---|
| No. | Ingredients | Qty/mg |
| 1. | Carnuba Wax | 5.00 |
| 2. | Iso Propyl Alcohol | q.s |
| 3. | Methylene chloride | q.s. |

Desloratadine and mannitol were cosifted to form Drug premix. The Drug premix was further blended with L-HPC, silica colloidal anhydrous and DC grade mannitol and this was further lubricated with siliconised talc and castor oil hydrogenated and compressed to form tablets. The tablets were coated with the above coating composition.

### EXAMPLE 13

Tablet by direct compression.

| Sr. No | Ingredients | Qty/Tab. (mg) |
|---|---|---|
| | Dry Mix | |
| 1. | Desloratadine (Form I) | 5.0 |
| 2. | Mannitol | 10.0 |
| 3. | Mannitol) (DC grade) | 96.5 |
| 4. | Low substituted hydroxy | 15.0 |
| | propyl cellulose (LHPC) | |
| 5. | Silica colloidal anhydrous) | 1.5 |
| | Lubrication | |
| 6. | Siliconised Talc (3% w/w) | 16.0 |
| 7. | Castor Oil Hydrogenated | 12.0 |
| | (Powder grade) | |
| | Total | 156.00 |

Coating

| Sr. No. | Ingredients | Qty/mg |
|---|---|---|
| 1. | Gum acacia | 17.00 |
| 2. | Sugar (Pharma grade) | 176.50 |
| 3 | Talc | 83.7 |
| 4 | Titanium dioxide | 2.8 |
| 5 | Purified Water | q.s. |
| | Polishing Coat Formula | |
| 6 | Carnuba Wax | 2.4 |
| 7 | Talc | 65.0 |

Desloratadine and mannitol were cosifted to form Drug premix. The Drug premix was further blended with L-HPC, silica colloidal anhydrous and DC grade mannitol and this was further lubricated with siliconised talc and castor oil hydrogenated and compressed to form tablets. The tablets were coated with the above coating composition.

The Stability of Formulated Tablets of Example 8

Chemical assay, physical properties and degradation of the formulated tablets of Example 8 were studied on samples placed in high density polyethylene bottles and blister packages. No significant changes were observed in the physical appearance, chemical assay of desloratadine and dissolution rate when the tablets of Example 8 were stored in High density polyethylene bottles (HDPE) or blister packages for up to 12 months at 25° C. 60% relative humidity (“RH”) or at 30° C./65% RH or for up to 6 months at 40° C./75% RH. A small amount of degradation e.g. N-formyl DCL, was observed in the tablets stored in HDPE bottles and in blisters (about 0.5%) at 40° C./75% RH for 6 months, and only about 0.2-0.3% was observed in samples of the tablets stored in blisters or HDPE bottles for 12 months at 25° C./60% or at 30° C./65% RH.

DC grade as used herein means directly compressible grade.

1. A stable pharmaceutical composition comprising desloratadine or a pharmaceutically acceptable salt, solvate, derivative, polymorph, hydrate or enantiomer thereof, and a carrier comprising at least one polyol selected from mannitol, maltitol, sorbitol, lactitol, erythritol, or xylitol or a mixture of two or more thereof, wherein the composition does not contain stearic acid and derivatives thereof.

2. The composition according to claim 1 wherein the total impurities amount to less than 1.5% by weight of the active.

3. The composition according to claim 1 which contains N-formyl desloratadine less than 0.75% by weight of the active.

4. The composition according to claim 1 which contains N-formyl desloratadine less than 0.6% by weight of the active.

5. The composition according to claim 1 which contains N-formyl desloratadine less than 0.5% by weight of the active.

6. The composition according to claim 2 wherein the composition has been stored at about 25° C. and about 60% relative humidity.

7. The composition according to claim 1 wherein the amount of polyol is from 1 to 95% by weight of the composition.

8. The composition according to claim 7 wherein the amount of polyol is from 15 to 85% by weight of the composition.

9. The composition according to claim 8 wherein the amount of polyol is from 50 to 80% by weight of the composition

10. The composition according to claim 1 further comprising at least one disintegrating agent.

11. The composition according to claim 10 wherein the amount of disintegrating agent is from 0.5 to 30% by weight of the composition.

12. The composition according to claim 10 wherein the disintegrating agent is starch, modified starch such as partially gelatinized starch, sodium starch glycolate, hydroxypropylcellulose, low substituted hydroxypropylcellulose or croscarmellose sodium or is a mixture of two or more thereof.

13. The composition according to claim 12 wherein the disintegrating agent is low substituted hydroxypropylcellulose.

14. The composition according to claim 1 further comprising an antioxidant and/or a chelating agent, or a mixture thereof.

15. The composition according to claim 14 wherein the amount of antioxidant or chelating agent is from 0.01 to 1% by weight of the composition.

16. The composition according to claim 15 wherein the amount of antioxidant or chelating agent is from 0.01 to 0.5% by weight of the composition.

17. The composition according to claim 14 wherein the antioxidant is sodium metabisulfite, butylated hydroxy anisole, butylated hydroxy toluene, propyl gallate, sodium ascorbate, or vitamin E acetate, or is a mixture of two or more thereof.

18. The composition according to claim 17 wherein the antioxidant is sodium metabisulfite.

19. The composition according to claim 1 comprising, in addition to desloratadine, from 50 to 80% polyol, from 5 to 15% disintegrating agent, and from 0.01 to 0.5% antioxidant and/or chelating agent, all by weight of the composition.

20. The composition according to claim 1 further comprising a lubricant.

21. The composition according to claim 20 wherein the lubricant is present in an amount of from 0.5 to 35% by weight of the composition.

22. The composition according to claim 20 wherein the lubricant is talc, hydrogenated castor oil, siliconised talc, anhydrous colloidal silica, sucrose esters, hydrogenated vegetable oil, silicone or simethicone oil, or is a mixture of two or more thereof.

23. The composition according to claim 1 which composition is coated.

24. The composition according to claim 23 wherein the coating is a film-coating, a sugar coating (other than lactose) or a wax coating.

25. The composition according to claim 23 which composition is film-coated.

26. The composition according to claim 25 wherein the film coat is free of lactose.

27. The composition according to claim 1 which composition is free of pharmaceutically acceptable basic salts.

28-33. (canceled)

34. The composition according to claim 1 which is a solid pharmaceutical composition.

35. (canceled)

36. A method comprising using a polyol selected from mannitol, maltitol, sorbitol, lactitol, erythritol or xylitol or a mixture of two or more thereof to stabilize a pharmaceutical composition comprising desloratadine, wherein the composition does not contain stearic acid and derivatives thereof.

37. The method according to claim 36 wherein the polyol is mannitol.

38. The composition according to claim 1 for use as a medicament.

39. A method comprising a composition according to claim 1 for the manufacture of a medicament for treating conditions responsive to administration of an antihistamine.

40. The process for making a stable pharmaceutical composition according to claim 1 which process comprises blending the ingredients and formulating them so as to form said composition.

41. The process for making a stable pharmaceutical tablet composition according to claim 1 acceptable excipients, which process comprises blending the ingredients and compressing to form a tablet.

42. The method of treating a condition responsive to administration of an antihistamine, which method comprises administering to a patient in need thereof a composition as claimed in claim 1.
